(19) United States
(12) Patent Application Publication
(10) Pub. No.: US 2003/0044398 A1
(43) Pub. Date: Mar. 6, 2003

(54) Methods for producing antibodies in mammals

(76) Inventors: James M. Robl, Belchertown, MA (US); Richard A. Goldsby, Leverett, MA (US)

Correspondence Address: CLARK & ELBING LLP, 101 FEDERAL STREET, BOSTON, MA, 02110, US

(21) Appl. No.: 10/095,875

(22) Filed: Mar. 20, 2002

Related U.S. Application Data
(60) Provisional application No. 60/277,460, filed on Mar. 20, 2001.

(51) Int. Cl. A61K 39/395

(52) U.S. Cl. 424/93.71; 530/387.1; 424/130.1

(57) ABSTRACT

The invention provides methods for producing antibodies by transplanting a cell that produces an antibody of interest into a mammal and isolating the desired antibodies from the mammal. The invention also features methods of transplanting antibody-producing cells into a mammal.

### CROSS-REFERENCE TO RELATED APPLICATION

This application claims the benefit of the filing date of U.S. provisional application No. 60/277,460, filed Mar. 20, 2001.

### BACKGROUND OF THE INVENTION

In general, the invention features methods for transplanting antibody-producing cells into mammals and methods for producing antibodies in mammals.

Antibodies may be used in a variety of research, diagnostic, therapeutic, and industrial applications. The production of antibodies by culturing hybridomas, genetically modified myeloma cells, or genetically modified CHO cells in vitro generally requires sophisticated and expensive equipment. In addition, the production of transgenic goats which secrete heterologous antibodies into milk is generally time-consuming and expensive. Bacterial and plant systems may also be used for the production of antibodies, but these systems are limited to nonglycosylated antibodies.

Thus, more rapid and less expensive methods are needed for the production of antibodies. Preferably, these methods produce little or no discomfort in the mammals that generate the antibodies.

### SUMMARY OF THE INVENTION

The purpose of the present invention is to provide improved methods for producing antibodies. In particular, these methods involve the transplantation of antibody-producing cells into a mammal and the isolation of the resulting antibodies from the mammal. In addition, improved methods have been developed for minimizing or preventing any adverse immune response to the foreign antibody-producing cells or the foreign antibodies secreted by these cells. These methods also reduce or eliminate the production of endogenous, undesired antibodies by the mammal. These improved methods involve chemically, serologically, or genetically modulating, reducing, or eliminating B-cell or T-cell activity in the mammal. Antibody-producing cells of the invention may also be administered to mammals (e.g., humans) for the treatment or prevention of a disease, disorder, or infection.

Accordingly, in a first aspect, the invention provides a method of producing antibodies. This method involves administering an antibody-producing cell from a donor source to a non-rodent recipient mammal in a site other than the peritoneal cavity and isolating the resulting antibodies from the recipient mammal. Preferably, the antibodies are isolated from the blood, milk, or lymph of the recipient mammal. The blood sample may be from any artery, vein, or capillary.

In a related aspect, the invention provides another method of producing antibodies. This method involves administering an antibody-producing cell from a donor source into an appropriate site in a recipient mammal during its embryonic or fetal stage and isolating the resulting antibodies from the embryo, fetus, or resulting offspring. Preferably, the antibodies are isolated from the blood, milk, or lymph of the recipient mammal. The blood sample may be from any artery, vein, or capillary. The antibody may be administered to the peritoneal cavity or to any site other than the peritoneal cavity.

In preferred embodiments of these antibody production methods, the immune system of the recipient mammal is suppressed, thereby reducing or preventing an adverse immune response in the recipient mammal to the foreign antibody-producing cell or the foreign antibodies. For example, a compound that inhibits B-cell activity or that destroys B-cells, such as an anti-IgM antibody, may be administered to the recipient mammal in an amount sufficient to reduce its B-cell activity. Alternatively, a compound that inhibits T-cell activity may be administered to the recipient mammal in an amount sufficient to reduce its T-cell activity. Examples of compounds that inhibit T-cell activity include cyclosporin, azathioprine, dexamethasone, anti-CD3 antibodies, anti-CD2 antibodies, and anti-CD25 antibodies. Examples of other preferred immunosuppressive agents that inhibit B-cell or T-cell activity include progesterone and polyclonal anti-lymphocyte globulin. Still other preferred immunosuppressive agents include steroid hormones (e.g., corticoids, glucocorticoids, or dexamethasone) and anti-inflammatory agents (e.g., Cox-1 inhibitors, Cox-2 inhibitors, aspirin, ibuprofen, vioxx, or celebrex). Compounds that inhibit B-cell activity, T-cell activity, or any other innate or adaptive immune system activity may be administered to the recipient mammal prior to, concurrent with, or after the transplantation of the antibody-producing cell or cells. These compounds may be administered during the normal period of development of the mammal's immune system (i.e., during the embryonic, fetal, or postnatal stage) or after this period of immune system development. Preferably, antibodies that inhibit B-cell or T-cell activity are a reactive against B-cells or T-cells in mammals of the same genus or species as the recipient mammal.

In still other preferred embodiments, the recipient mammal is tolerized by exposure to antigens similar to the foreign antibody-producing cell or the foreign antibodies produced by this cell, thereby reducing or preventing an adverse immune response in the recipient mammal after administration of the foreign antibody-producing cell. In one embodiment, a cell (e.g., a fetal cell or a bone marrow cell such as a hematopoietic stem cell) of the same genus or species as the donor source is administered to the recipient mammal during the normal period of development of the mammal's immune system, such as during the mammal's embryonic, fetal, or postnatal stage. Alternatively, a protein from a cell, embryo, fetus, or mammal of the same genus or species as the donor source is administered to the recipient mammal during the normal period of development of the mammal's immune system. Preferred proteins include serum proteins, such as IgM, IgD, IgG, IgE, or IgA. The foreign cells or proteins may be administered in a single dose or in multiple doses (e.g., 1, 2, 3, or more doses per day, week, or month). In preferred embodiments, multiple does are administered throughout the normal period of development of the mammal's immune system or until the mammal is no longer used for the production of antibodies. The cells or proteins may optionally be administered with a pharmaceutically acceptable diluent, carrier, or excipient, such as saline, buffered saline, dextrose, water, glycerol, ethanol, or a combination thereof.

In other preferred embodiments of the antibody production methods, the recipient mammal is a chimeric mammal that includes cells of the same genus or species as the donor source and cells from another genus or species. In still other preferred embodiments, the recipient mammal possesses a heterozygous or homozygous mutation that reduces or eliminates the expression or activity of IgM, IgD, IgG, IgE, IgA, recombinase activating genes 1 (RAG1), or RAG2 (RAG2). These mutations may reduce or eliminate the production of endogenous antibodies by the recipient mammal and thereby facilitate the isolation of the foreign antibody of interest that is produced by the administered antibody-producing cell or cells.

The invention also provides methods of transplanting an antibody-producing cell into a recipient mammal that has a reduced immune response to the antibody-producing cell or to the antibodies produced by the cell. For example, the recipient mammal may be tolerized by exposure to cells or proteins that are similar to the antibody-producing cell or to the antibodies produced by the cell. Alternatively, the immune system of the recipient mammal may be suppressed by chemical or serological inhibition of an immune system activity. Other preferred recipient mammals have a naturally-occurring or engineered mutation that reduces immune system activity. These methods are useful for minimizing any adverse immune response to the foreign cell or antibodies and for facilitating antibody secretion into the bloodstream.

According to this aspect of the invention, a method is provided for transplanting an antibody-producing cell into a recipient mammal. This method involves tolerizing the recipient mammal to the antibody-producing cell or the antibodies produced by the antibody-producing cell and administering the antibody-producing cell to the recipient mammal. In one preferred embodiment, the antibody-producing cell is obtained from a donor source of a different genus or species as the recipient mammal. A preferred tolerization step involves administering a cell of the same genus or species as the donor source to the recipient mammal during the normal period of development of the mammal's immune system (e.g., the embryonic, fetal, or postnatal stage). Preferred cells that may be administered include bone marrow cells, such as hematopoietic stem cells, and fetal cells. In another preferred embodiment, the tolerization step includes administering a protein from a cell, embryo, fetus, or mammal of the same genus or species as the donor source to the recipient mammal during the normal period of development of the mammal's immune system. Preferred proteins include serum proteins. The foreign cells or proteins may be administered in a single dose or in multiple doses (e.g., 1, 2, 3, or more doses per day, week, or month). In preferred embodiments, multiple does are administered throughout the normal period of development of the mammal's immune system or until the mammal is no longer used for the production of antibodies.

In a related aspect, the invention provides another method for transplanting an antibody-producing cell into a recipient mammal. This method involves suppressing the immune system of the recipient mammal and administering an antibody-producing cell to the recipient mammal. A preferred immunosuppression step involves administering a compound that inhibits B-cell activity, such as an anti-IgM antibody, to the recipient mammal in an amount sufficient to reduce its B-cell activity. Another preferred immunosuppression step involves administering a compound that inhibits T-cell activity to the recipient mammal in an amount sufficient to reduce its T-cell activity. Preferably, the compound is cyclosporin, azathioprine, dexamethasone, an anti-CD3 antibody, an anti-CD2 antibody, or an anti-CD25 antibody. Examples of other preferred immunosuppressive agents that inhibit B-cell or T-cell activity include progesterone and polyclonal anti-lymphocyte globulin. Still other preferred immunosuppressive agents include steroid hormones (e.g., corticoids, glucocorticoids, or dexamethasone) and anti-inflammatory agents (e.g., Cox-1 inhibitors, Cox-2 inhibitors, aspirin, ibuprofen, vioxx, or celebrex). In yet another preferred embodiment, a compound that inhibits B-cell activity, T-cell activity, or any other innate or adaptive immune system activity is administered to the recipient mammal during the normal period of development of the mammal's immune system (i.e., during its embryonic, fetal, or postnatal stage) or after this period. An immunosuppressive agent may also be administered to the recipient mammal prior to, concurrent with, or after the transplantation of the antibody-producing cell or cells. In a preferred embodiment, a human antibody-producing cell is administered to a human in need of the antibodies produced by the antibody-producing cell. This transplantation method can be used for the treatment or prevention of a disease, disorder, or infection in a mammal (e.g., a human).

In another related aspect, the invention provides yet another method of transplanting an antibody-producing cell into a recipient mammal. This method involves administering the antibody-producing cell to a recipient mammal that is a chimeric mammal having both cells of the same genus or species as the antibody-producing cell and cells of a different genus or species as the antibody-producing cell. In one preferred embodiment, the chimeric mammal is generated by administering cells of the same genus or species as the antibody-producing cell to the recipient mammal during its embryonic or fetal stage.

In yet related aspect, the invention provides another method of transplanting an antibody-producing cell into a recipient mammal. This method involves administering the antibody-producing cell to a recipient mammal that has a homozygous or heterozygous mutation that reduces or eliminates the expression or activity of IgM, IgD, IgG, IgE, IgA, RAG1, or RAG2. The antibody-producing cell may be administered subcutaneously or non-subcutaneously.

The antibody-producing cells of the invention may also be administered to a mammal for the treatment or prevention of a disease, disorder, or infection. For example, an antibody-producing cell may be administered to a mammal diagnosed with, or at increased risk for, a disease or disorder associated with a lower than normal level of a particular antibody. Alternatively, a cell producing an antibody reactive with a toxin or an infectious agent (e.g., a virus, bacteria, yeast, or parasite) may be administered for the treatment or prevention of an infection. Moreover, a cell that produces an antibody reactive with an antigen produced by a cancerous cell may be administered for the treatment or prevention of cancer. As an alternative to administering an antibody-producing cell, a nucleic acid encoding an antibody of interest may be administered to a mammal to modify a cell in vivo, resulting in the expression of the desired antibody by the genetically modified cell.

In one such aspect, the invention provides a method of treating or preventing a disease, disorder, or infection in a mammal. This method involves administering an antibody-producing cell to a mammal in any appropriate site that results in the production of antibodies by the antibody-producing cell. In one embodiment, a differentiated or undifferentiated cell (e.g., a fibroblast cell or stem cell) obtained from the mammal is genetically modified by the insertion of a nucleic acid encoding a desired antibody and then re-administered to the same mammal. In another embodiment, the cell is modified by the addition of an oncogene to increase the length of time the cell may be maintained ex vivo and to increase the time during which the cell may be modified to contain a nucleic acid encoding the antibody. If desired, the oncogene may be removed from the cell before it is re-administered to the mammal. The cell may be administered subcutaneously, intramuscularly, or by any other appropriate route of administration to any appropriate site in the mammal. Preferably, the desired antibody is secreted by the re-administered cell or progeny of the re-administered cell for at least 1, 10, 20, 40, 60, 80, 100, or more weeks. In other preferred embodiments, the antibody is reactive with a toxin, a pathogen (e.g., a virus, bacteria, yeast, or parasite), or an antigen expressed on the surface of a cancerous cell. For example, cells producing an anti-RSV antibody or an anti-CD20 antibody may be administered for the treatment, stabilization, or prevention of RSV infection or HIV infection, respectively. Additionally, cells producing an anti-IgE antibody may be used for the treatment or prevention of an allergy, and cells producing an antibody reactive with the inflammatory cytokine TNF may be used for the treatment or prevention of rheumatiod arthritis or any other inflammatory condition. It is also contemplated that a cell producing an antibody that increases an activity of a protein (e.g., an enzyme, hormone, antibody, or cell receptor) may be used to treat, stabilize, or prevent conditions involving decreased or insufficient activity of the protein, and a cell producing an antibody that decreases an activity of a protein may be used to treat, stabilize, or prevent conditions involving increased or undesirable activity of the protein.

In a related aspect, the invention provides another method of treating or preventing a disease, disorder, or infection in a mammal. This method involves genetically modifying a cell in a mammal by administering a nucleic acid encoding a desired antibody into the mammal. In preferred embodiments, the nucleic acid encoding the desired antibody is operably linked to a promoter and contained in an expression vector (e.g., a plasmid or a recombinant viral vector, such as an adenoviral, adeno-associated viral, retroviral, lentiviral, herpes viral vector, or baculovirus-based system). In other preferred embodiments, the nucleic acid is administered intravenously in combination with a liposome and protamine. The nucleic acid may also be administered subcutaneously, intramuscularly, or by any other appropriate route of administration to any appropriate site in the mammal. In preferred embodiments, the nucleic acid is administered to, or expressed in, a mammary gland, uterus, scrotum, or testicle of the mammal. Preferably, the desired antibody is secreted by the genetically-modified cell or progeny of the genetically-modified cell for at least 1, 10, 20, 40, 60, 80, 100, or more weeks. In other preferred embodiments, the antibody is reactive with a toxin, pathogen (e.g., a virus, bacteria, yeast, or parasite), or antigen expressed on the surface of a cancerous cell. For example, a nucleic acid encoding an anti-RSV antibody or an anti-CD20 antibody may be administered for the treatment, stabilization, or prevention of RSV infection or HIV infection, respectively. Additionally, a nucleic acid encoding an anti-IgE antibody may be used for the treatment or prevention of an allergy, and a nucleic acid encoding an antibody reactive with TNF may be used for the treatment or prevention of rheumatiod arthritis or any other inflammatory condition. It is also contemplated that nucleic acids encoding an antibody that increases an activity of a protein (e.g., an enzyme, hormone, antibody, or cell receptor) may be used to treat, stabilize, or prevent conditions involving decreased or insufficient activity of the protein, and nucleic acids encoding an antibody that decreases an activity of a protein may be used to treat, stabilize, or prevent conditions involving increased or undesirable activity of the protein.

In preferred embodiments of various aspects of the invention, the antibody-producing cell is administered to a mammary gland, uterus, dewlap, brisket, scrotum, testicle, or hump (e.g., a hump on the back of a cow or camel) of the recipient mammal. The antibody-producing cell may be administered subcutaneously or non-subcutaneously. Other suitable sites include any area that maintains the viability of the antibody-producing cell or progeny of the antibody-producing cell and that allows the secreted antibodies to enter the bloodstream. Preferably, the administration of the antibody-producing cell does not elicit the production of a substantial amount of ascites fluid. In various embodiments, the antibody-producing cell is administered to a site other than the peritoneal cavity.

Preferably, the administration of the antibody-producing cell does not cause chronic pain or inflammation in the recipient mammal. Preferably, any signs of inflammation (e.g., redness, swelling, heat, or ascites fluid) located at, or proximal to, the site of injection last less than 20, 15, 10, 5, 3, or 1 day. In still other preferred embodiments, inflammation induced by the administration of an antibody-producing cell lasts less than 20, 15, 10, 5, 3, or 1 day longer than administration of a vehicle only control. Preferably, an adjuvant or irritant is not administered to the recipient mammal.

If desired, the antibody-producing cell may be administered in a pharmaceutically acceptable diluent, carrier, or excipient such as saline, buffered saline, dextrose, water, glycerol, ethanol, or a combination thereof. The antibody-producing cell may be administered during the fetal, embryonic, or postnatal stage of the recipient mammal. In preferred embodiments, at least 10, 10², 10³, 10⁶, 10⁸, 10⁹, or more antibody-producing cells are administered to the recipient.

In various aspects of the invention, the recipient mammal or the donor source may be a human or non-human mammal and is preferably a member of the genus Bos. Examples of other preferred recipient mammals and donor sources include cows, sheep, big-horn sheep, goats, buffalos, antelopes, oxen, horses, donkeys, mule, deer, elk, caribou, water buffalo, camels, llama, alpaca, rabbits, pigs, mice, rats, guinea pigs, hamsters, primates such as monkeys, and birds such as chickens and turkeys. The recipient mammal may be isogenic or non-isogenic to the antibody-producing cell. In preferred embodiments, the recipient mammal is less than 50, 40, 30, 20, 10, 7, 5, 4, 3, 2, or 1 week old. In yet other preferred embodiments, a fetus is allowed to develop until a chosen time in a pregnant or host mammal, and then the fetus is surgically removed or labor is induced using standard methods. For example, a viable fetus may be removed by Caesarian section, or labor may be artificially induced 1, 2, 3, 5, 10, 15, 20, or more days prior to the normal term of the fetus. These young recipient mammals may have a naturally suppressed immune system, thereby minimizing or preventing an adverse immune response to the foreign antibody-producing cell or antibodies. Other preferred mammals naturally or spontaneously have an immune system that is less responsive than normal.

Another preferred recipient mammal is a cloned mammal that has an identical or substantially identical genome as the antibody-producing cell. This cloned mammal may be produced by transferring a cell or nucleus from a donor source into an enucleated oocyte. The oocyte or an embryo formed from the oocyte is transferred to the uterus of a host mammal under conditions that allow the oocyte or the embryo to develop into a live offspring. The resulting cloned mammal is less likely to reject transplanted antibody-producing cells having an identical or substantially identical genome as the donor source used to generate the cloned mammal. It is also contemplated that an antibody-producing cell may be injected into a pre-implantation embryo or into a fetus to generate a chimeric mammal that produces the antibody of interest.

The antibody-producing cell used in any of the above methods may be an immortalized or a non-immortalized cell and may be obtained from a donor source of the same species or of a different genus or species as the recipient mammal. Preferred non-immortalized cells include primary cells that are obtained from the recipient mammal, genetically modified to produce the desired antibody, and re-administered to the recipient mammal. Antibody-producing cells may also be genetically modified by the insertion of heavy or light chain antibody genes that encode an antibody of interest or by the insertion of genes that facilitate the immortalization of the cells.

The antibodies produced by these methods may be monoclonal or polyclonal. If desired, two or more antibody-producing cells may be administered to the same recipient mammal for the production of two or more different antibodies in that mammal. Preferred antibodies bind RSV, CMV, CD20, CD25, CD33, TNF, or Her2. Other preferred antibodies are human or humanized. The antibody may also originate in another genus or species. In other preferred embodiments, the antibody is a bifunctional antibody. Still other preferred antibodies include those having, or consisting of, a ScFv, Fab, or F(ab′)₂ fragment. Other examples of preferred antibodies include derivatized antibodies encoded by a fusion nucleic acid that has been modified through gene fusion technology so that the nucleic acid encoding the antibody or a fragment of the antibody is operably linked to a nucleic acid encoding a toxin, therapeutically active compound, enzyme, cytokine, or affinity tag. The covalently linked group in the derivatized antibody many be attached to the amino-terminus, carboxy-terminus, or between the amino- and carboxy-termini, of the antibody or antibody fragment. By “affinity tag” is meant a peptide, protein, or compound that binds another peptide, protein, or compound. In a preferred embodiment, the affinity tag is used for purification or immobilization of the derivatized antibody. In another preferred embodiment, the affinity tag or toxin is used in therapeutic applications of the antibody to target the antibody to a specific cell, tissue, or organ system in vivo. In yet another preferred embodiment, the therapeutically active compound is used for the treatment or prevention of a disease or disorder. It is also contemplated that antibodies isolated from recipient mammals may be subsequently chemically modified so that they are covalently linked to a toxin, therapeutically active compound, enzyme, cytokine, radiolabel, fluorescent label, or affinity tag. If desired, the fluorescent or radiolabel may be used for imaging of the antibody in vitro or in vivo.

As used herein, by “donor source” is meant the source from which an antibody-producing cell or cloned mammal is derived. Examples of donor sources include, but are not limited to, embryos, fetuses, mammals, cell lines, and other cells cultured in vitro.

By “antibody-producing cell” is meant a cell that naturally produces an antibody, such as a B-cell, or a cell that is genetically modified to encode or express an antibody that binds an antigen of interest. Examples of cell types that may be modified to produce antibodies include differentiated cells, such as epithelial cells, neural cells, epidermal cells, keratinocytes, hematopoietic cells, melanocytes, chondrocytes, T-cells, erythrocytes, macrophages, monocytes, fibroblasts, and muscle cells; and undifferentiated cells, such as embryonic or adult stem cells. Other preferred antibody-producing cells include myeloma cells (e.g. SP20 cells, NSO cells, or myelomas produced from transformed blast cells) and spontaneously or artificially immortalized cell lines (e.g. CHO cells). In preferred embodiments, the cells are from a cloned mammal that has an identical or substantially identical genome as the recipient mammal. The donor cells may be transfected with a mammalian expression vector that includes a promoter operably-linked to a nucleic acid encoding an antibody. Examples of suitable promoters include those derived from immunoglobulin genes, SV40, adenovirus, bovine papiloma virus, or cytomegalovirus (see, for example, U.S. Pat. Nos. 6,057,098 and 5,789,208). The nucleic acids encoding the antibody may include known sequences, mutagenized sequences, or sequences selected using standard selection methods for isolating proteins that bind an antigen of interest, such as mRNA display (see, for example, Publication Number WO 98/31700), ribosome display (see, for example, Roberts, Curr. Opin. Chem. Biol. 3(3):268-73, 1999), or phage display (see, for example, U.S. Pat. Nos. 5,821,047 and 5,658,727). Additionally, the nucleic acid may comprise a mutation that increases the stability or solubility of the encoded antibody or that causes the antibody to elicit a less severe adverse immune response when produced in the recipient mammal or when administered to mammals, such as humans, for therapeutic or diagnostic applications.

By “mutation” is meant an alteration in a naturally-occurring or reference nucleic acid sequence, such as an insertion, deletion, frameshift mutation, silent mutation, nonsense mutation, or missense mutation. Preferably, the amino acid sequence encoded by the nucleic acid sequence has at least one amino acid alteration from a naturally-occurring sequence. Examples of recombinant DNA techniques for altering the genomic sequence of a cell, embryo, fetus, or mammal include inserting a DNA sequence from another organism (e.g., a human) into the genome, deleting one or more DNA sequences, and introducing one or more base mutations (e.g., site-directed or random mutations) into a target DNA sequence. Examples of methods for producing these modifications include retroviral insertion, artificial chromosome techniques, gene insertion, random insertion with tissue specific promoters, homologous recombination, gene targeting, transposable elements, and any other method for introducing foreign DNA. All of these techniques are well known to those skilled in the art of molecular biology (see, for example, Ausubel et al., supra).

By “immortilized” is meant capable of undergoing at least 25, 50, 75, 90, or 95% more cell divisions than a naturally-occurring control cell of the same cell type, genus, and species as the immortalized cell. Preferably, an immortalized cell is capable of undergoing at least 2, 5, 10, or 20-fold more cell divisions than the control cell. More preferably, the immortalized cell is capable of undergoing an unlimited number of cell divisions. Examples of immortalized cells include cells that naturally acquire a mutation in vivo or in vitro that alters their normal growth-regulating process. Other preferred immortalized cells include hybridoma cells which are generated using standard techniques for fusion of a myeloma with a B-cell (Mocikat, J. Immunol. Methods 225:185-189, 1999; Jonak et al., Hum. Antibodies Hybridomas 3:177-185, 1992; Srikumaran et al., Science 220:522, 1983). Preferred hybridomas include those generated from the fusion of a B-cell from the donor source with a myeloma from a mammal of the same genus or species as the recipient mammal. Other preferred myeloma or B-cells are from a Balb/C mouse or a human. Still other preferred immortalized cells include cells that have been genetically modified to express an oncogene, such as ras, myc, abl, bcl2, or neu, or that have been infected with a transforming DNA or RNA virus, such as Epstein Barr virus or SV40 virus (Kumar et al., Immunol. Lett. 65:153-159, 1999; Knight et al., Proc. Nat. Acad. Sci. USA 85:3130-3134, 1988; Shammah et al., J. Immunol. Methods 160-19-25, 1993; Gustafsson and Hinkula, Hum. Antibodies Hybridomas 5:98-104, 1994; Kataoka et al., Differentiation 62:201-211, 1997; Chatelut et al., Scand. J. Immunol. 48:659-666, 1998).

By “non-immortilized” is meant not immortalized as described above.

By “isolating antibodies” is meant purifying antibodies from a sample obtained from a recipient mammal. The antibodies may be purified by one skilled in the art using standard techniques such as those described by Ausubel et al. (Current Protocols in Molecular Biology, volume 2, p. 11.13.1-11.13.3, John Wiley & Sons, 1995). Preferred methods of purification include precipitation using antigen or antibody coated beads, column chromatography such as affinity chromatography, magnetic bead immunoaffinity purification, and panning with a plate-bound antigen. The antibody is preferably at least 2, 5, or 10 times as pure as the starting sample, as measured using polyacrylamide gel electrophoresis, column chromatography, optical density, HPLC analysis, or western analysis to detect a reduction in the amount of contaminating proteins or ELISA to detect an increase in specific activity for binding to an antigen of interest (Ausubel et al., supra). In another preferred embodiment, the antibody is at least 75%, more preferably, at least 90%, and most preferably, at least 99%, by weight, pure.

By “inhibiting B-cell activity” is meant reducing the amount of antibodies produced by a B-cell or a population of B-cells. This reduction in the amount of antibodies may be due to a decrease in the amount of antibodies produced per B-cell, a decrease in the number of functional B-cells, or a combination thereof. Preferably, the amount of an antibody secreted by a B-cell or expressed on the surface of a B-cell is reduced by at least 25, 50, 75, 90, or 95%. In another preferred embodiment, the number of B-cells in a sample from the recipient mammal, such as a blood sample, is reduced by at least 25, 50, 75, 90, or 95%.

By “inhibiting T-cell activity” is meant reducing an immune response of a T-cell or a population of T-cells. This reduction in the immune response may be due to a decrease in the activity of the T-cells, a decrease in the amount of functional T-cells, or a combination thereof. The decrease in the ability of cytotoxic or “natural killer” T cells to destroy foreign cells may be measured using standard assays. Preferably, this cytotoxicity is reduced by at least 25, 50, 75, 90, or 95%. In another preferred embodiment, the production of a cytokine, such as IL-2, IL-4, IL-5, IL-6, IL-9, IL-10, or IL-13, is reduced by at least 25, 50, 75, 90, or 95%. In another preferred embodiment, the number of T-cells in a sample from the recipient mammal, such as a blood sample, is reduced by at least 25, 50, 75, 90, or 95%. In yet another preferred embodiment, the number of T-cells that are present in the sample after incubation with a mitogen is reduced by at least 25, 50, 75, 90, or 95% compared to a similarly treated sample from a recipient mammal that has a not been administered a compound which inhibits T-cell activity.

By “mammal with an immune system that is less responsive than normal” is meant a recipient mammal that naturally or spontaneously has an innate or adaptive immune system that is less active than normal. For example, the recipient mammal may have fewer B-cells, fewer Ig molecules expressed on the surface of B-cells, fewer antibody molecules secreted by B-cells, fewer T-cells, fewer cytokine molecules produced by T-cells exposed to an antigen or mitogen, less cytotoxic activity or proliferation of T-cells in response to an antigen or mitogen, or fewer antibody or cytokine molecules produced in response to administration of an antigen, based on standard methods such as those described herein. Preferably, the number of any of the above cells or immunoglobulins or the level of any of the above activities in a recipient mammal is less than the average number of cells or immunoglobulins or the average level of activity for mammals of the same genius, species, and age. In other preferred embodiments, the number of any of these cells or immunoglobulins or the level of any of these activities in a recipient mammal is less 90, 80, 70, 60, 50, 40, 30, or 20% of the corresponding number of cells or immunoglobulins or the corresponding level of activity in another mammal of the same genus, species, and age. Any of these assays may also be used to identify the mammals in a population of potential recipient mammals with the least active immune systems.

By “embryo” or “embryonic” is meant a developing cell mass that has not implanted into the uterine membrane of a maternal host. Hence, the term “embryo” may refer to a fertilized oocyte, an oocyte containing a donor nucleus, a pre-blastocyst stage developing cell mass, or any other developing cell mass that is at a stage of development prior to implantation into the uterine membrane of a maternal host and prior to formation of a genital ridge. An embryo may represent multiple stages of cell development. For example, a one cell embryo can be referred to as a zygote; a solid spherical mass of cells resulting from a cleaved embryo can be referred to as a morula, and an embryo having a blastocoel can be referred to as a blastocyst.

By “fetus” or “fetal” is meant a developing cell mass that has implanted into the uterine membrane of a maternal host. A fetus may have defining features such as a genital ridge which is easily identified by a person of ordinary skill in the art.

By “ascites fluid” is meant serous fluid in the peritoneal cavity. The accumulation of this fluid may result from inflammation of the peritoneal cavity or the membrane lining the peritoneal cavity.

By “not eliciting the production of a substantial amount of ascites fluid” is meant not inducing the production or accumulation of a substantial amount of ascites fluid in the peritoneal cavity. Preferably, the amount of ascites fluid in the peritoneal cavity increases by less than 20, 10, 5, 3, or 2-fold, as measured using standard methods. More preferably, the amount of ascites fluid in the peritoneal cavity increases by less than 100, 75, 50, 25, 10, or 5%. In other preferred embodiments, the volume of ascites fluid in the peritoneal cavity is less than 150, 100, 75, 50, 25, 10, 5, 3, or 1 mL. Most preferably, no ascites fluid is produced, or no ascites fluid remains 1, 2, 3, or 5 days after administration of the antibody-producing cell. In one possible method for measuring the volume of ascites fluid in the peritoneal cavity, a needle (e.g., an 18-gauge needle) attached to a syringe is inserted into the peritoneal cavity and used to withdraw the ascites fluid from the mammal (see, for example, Ausubel et al. Current Protocols in Molecular Biology, John Wiley & Sons, New York, 2000). The volume of the harvested ascites fluid is then determined.

By “humanized” is meant having an altered amino acid sequence so that fewer antibodies and/or immune responses are elicited against the humanized antibody when it is administered to a human. For example, the constant region of the antibody may be replaced with the constant region from a human antibody. For the use of the antibody in a mammal other than a human, an antibody may be converted to that species format.

By “bifunctional antibody” is meant an antibody that includes an antibody or a fragment of an antibody covalently linked to a different antibody or a different fragment of an antibody. In one preferred embodiment, both antibodies or fragments bind to different epitopes expressed on the same antigen. Other preferred bifunctional antibodies bind to two different antigens. Standard molecular biology techniques such as those described herein may be used to operably link two nucleic acids so that the fusion nucleic acid encodes a bifunctional antibody.

By “fragment” is meant a polypeptide having a region of consecutive amino acids that is identical to the corresponding region of an antibody of the invention but is less than the full-length sequence. The fragment has the ability to bind the same antigen as the corresponding antibody based on standard assays, such as those described herein. Preferably, the binding of the fragment to the antigen is at least 20, 40, 60, 80, or 90% of that of the corresponding antibody.

By “substantially identical” is meant having a genome that is at least 60, 70, 80, 90, 95, or 100% identical to that of another genome. Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various substitutions, deletions, and other modifications.

The present invention provides a number of advantages related to the production of antibodies. For example, hundreds of grams to kilograms of antibody may be produced in a short period of time at low cost. In addition, the administration of antibody-producing cells to a mammary gland, uterus, dewlap, brisket, scrotum, testicle, or hump of the recipient mammal does not require an adjuvant or irritating carrier and does not physically displace organs or muscles. Thus, the administration of these cells is minimally invasive and causes little or no discomfort. If desired, any discomfort may be further reduced by using a local anesthetic. Because hybridoma cells grow as solid, noninvasive masses, they should also cause minimal chronic pain or discomfort.

Other features and advantages of the invention will be apparent from the following detailed description and from the claims.

### BRIEF DESCRIPTION OF THE DRAWING

FIG. 1A is a schematic diagram of the bovine RAG2 knockout construct. FIG. 1B is the polynucleotide sequence of a portion of the knockout construct containing part of the ˜1.5 Kb bovine sequence immediately upstream of the RAG2 coding sequence, the bovine RAG2 coding sequence, and the ˜0.3 Kb bovine sequence immediately downstream of the RAG2 coding sequence. The two possible ATG start codons for the RAG2 coding sequence and the TTT stop codon are listed in bold font. The RAG2 coding sequence was modified by the insertion of the neomycin resistance gene in the opposition orientation (3′ to 5′). FIG. 1C is a polynucleotide sequence containing bovine sequence upstream of the RAG2 coding sequence, bovine RAG2 coding sequence, and bovine sequence immediately downstream of the RAG2 coding sequence. Nucleotides 3313 to 3368 are from the phage vector containing this RAG-2 sequence.

FIGS. 2A-2J are pictures of FACS analysis of peripheral blood lymphocytes from either experimental fetuses injected with an anti-bovine IgM antibody (das6) to inhibit B-cell development or control fetuses. FIGS. 2A-2E are pictures of the FACS analysis performed using an anti-bovine IgM antibody to detect IgM molecules expressed on the surface of B-cells. As illustrated in FIG. 2A and FIG. 2B, approximately 19.82 to 26.61% of the peripheral blood lymphocytes from the control fetuses expressed IgM. In contrast, 7.78, 11.80, or 3.95% of the peripheral blood lymphocytes from the three fetuses injected with the anti-bovine IgM antibody expressed IgM (FIGS. 2C-2E, respectively). FIGS. 2F-2J are pictures of the FACS analysis performed using an anti-bovine light chain antibody (das10) to detect antibody light chain molecules expressed on the surface of B-cells. As illustrated in FIG. 2F and FIG. 2G, approximately 12.43 to 29.47% of the peripheral blood lymphocytes from the control fetuses expressed antibody light chain molecules. In contrast, 2.54, 13.77, or 3.99% of the peripheral blood lymphocytes from the three fetuses injected with the anti-bovine IgM antibody expressed antibody light chain molecules (FIGS. 2H-2J, respectively).

### DETAILED DESCRIPTION

We have developed an improved method for generating antibodies of interest in mammals. In this antibody-production system, an antibody-producing cell, such as a hybridoma, is administered to a mammal in a site such as the mammary gland, uterus, dewlap, brisket, scrotum, testicle, or hump of the mammal during its embryonic, fetal, postnatal, or adult stage. These sites allow growth of the benign transplant to take place without discomfort to the animal. The high level of vascularization of a mammary gland and uterus facilitates the secretion of the desired antibodies into the bloodstream of the mammal. The antibodies may subsequently enter other fluids, such as milk or lymph, by passive or active diffusion from the bloodstream. The lower level of vascularization of the dewlap still allows the secreted antibodies to reach the bloodstream, while minimizing any adverse immune response to the transplanted antibody-producing cell. In preferred embodiments, the antibody-producing cell is not administered to the peritoneal cavity of the mammal because this site of administration may result in substantial inflammation, discomfort, or ascites fluid in the mammal. Blood, milk, or lymph is obtained from the mammals, and the desired antibody is purified from the fluid using standard protein purification techniques. Large mammals, such as cattle, goats, pigs, and horses, are desirable host animals because they tolerate the growth of a large number of transplanted antibody-producing cells. In addition, the long life span of these mammals increases the amount of antibody that may be obtained from them.

This method was used to produce an anti-tetanus antibody in the bloodstream of a calf. For this procedure, cells from a mouse hybridoma that secretes an anti-tetanus antibody were injected into the dewlap and mammary region of a calf. The mouse anti-tetanus antibody was detected in blood samples from the calf. The anti-tetanus antibody maintained its ability to bind tetanus toxin, demonstrating that functional antibodies may be produced using this method.

This method was also used to produce an anti-tetanus antibody in the ascites fluid and bloodstream of fetuses in pregnant cattle. The fetuses were removed from the pregnant cattle, and the mouse hybridoma cells described above were injected into the peritoneal cavity. The fetuses were then returned to the pregnant cattle to allow growth of the hybridoma cells. The mouse anti-tetanus antibody was later detected in ascites fluid and cord blood from the fetuses, indicating that functional antibodies can also be produced in fetuses using the method described herein.

The invention also features improved methods for transplanting antibody-producing cells into mammals. These methods involve either tolerizing a mammal to the foreign antibody-producing cell or the antibodies produced by the cell, suppressing the immune system of a mammal, or generating an immunodeficient mammal. An antibody-producing cell is then administered to the tolerized, immunosuppressed, or immunodeficient mammal.

In preferred tolerization methods, proteins or cells of the same genus or species as the ultimately transplanted antibody-producing cell are administered to an embryo or fetus to reduce or prevent an immune response when the antibody-producing cell is administered to the embryo, fetus, or resulting offspring. Alternatively, for mammals that are born with an immature immune system, the proteins or cells may be administered postnatally during the remainder of the developmental stage of the immune system. To maintain tolerance, the administration of the proteins or cells may be continued after the developmental stage of the immune system until the mammal is no longer used for the production of the antibody of interest. Other tolerized recipient mammals include chimeric mammals that have cells of the same genus or species as the subsequently transplanted antibody-producing cells and that have cells of a different genus or species.

To suppress the immune system of a recipient mammal, a compound that reduces the production of antibodies by B-cells or that reduces the number of active B-cells may be administered to the mammal. For example, we demonstrated that the injection of an anti-bovine IgM antibody into fetuses reduced the number of peripheral blood B-cells. Other preferred immunosuppression methods include the administration of a compound to a mammal that decreases an immune response by T-cells, the proliferation of T-cells, or the number of active T-cells. These immunosuppressive compounds may be administered before, concurrent with, or after the transplantation of the antibody-producing cell. These compounds may also be administered during the normal period of development of the mammal's immune system (e.g., during the fetal, embryonic, or postnatal stage) or after this period to inhibit the development of B-cells or T-cells in the mammal Examples of methods for generating immunodeficient mammals include the use of standard molecular biology techniques to produce a mammal that has a mutation in an IgM, IgD, IgG, IgE, IgA, RAG1, or RAG2 gene. These mutations may reduce or prevent the development of functional B-cells or T-cells.

The antibodies produced using the methods of the invention may be utilized in a variety of applications. For example, the antibodies may be used in immunoaffinity chromatography or immunopreciptation methods to purify an antigen of interest. In addition, these antibodies may be used in diagnostic methods to visualize or quantify the amount of a particular antigen in vivo or in vitro. The antibodies may also be used in therapeutic applications that involve the administration of an antibody to increase or decrease an activity associated with a disease or disorder. For example, antibodies that bind and inactivate a toxin secreted by a bacterial pathogen may be used to treat or prevent a disease caused by the pathogen. Additionally, antibodies that are covalently-labeled with a toxin may be used to target the toxin to cancer cells for the treatment, stabilization, or prevention of cancer.

The invention also provides methods of treating or preventing a disease, disorder, or infection in a mammal by administering an antibody-producing cell to a mammal in any appropriate site that results in the production of antibodies by the antibody-producing cell. For example, a differentiated or undifferentiated cell (e.g., a fibroblast cell or stem cell) obtained from the mammal is genetically modified using standard methods to insert a nucleic acid encoding a desired antibody. This genetically modified cell is then re-administered to the same mammal. If necessary, to increase the length of time the cell may be modified ex vivo to contain a nucleic acid encoding the desired antibody, the cell may be modified by the transient transfection of a plasmid containing an oncogene flanked by loxP sites for the Cre recombinase and containing a nucleic acid encoding the Cre recombinase under the control of an inducible promoter (Cheng et al., Nucleic Acids Res. 28(24):E108, 2000). The insertion of this plasmid results in the controlled immortalization of the cell. After the cell is modified to contain the nucleic acid encoding the desired antibody and is ready to be re-administered to the mammal, the loxP-oncogene-loxP cassette may be removed from the plasmid by the induction of the Cre recombinase which causes site specific recombination and loss of the cassette from the plasmid. Due to the removal of the cassette containing the oncogene, the cell is no longer immortalized and may be re-administered to the mammal without causing the formation of a cancerous tumor.

Other methods of treating or preventing a disease, disorder, or infection in a mammal involve administering a nucleic acid encoding an antibody of interest to the mammal. The nucleic acids may be delivered by gene therapy using a means such as viral vectors. In this method, a nucleic acid is cloned into the genome of a recombinant virus, such as an adenoviral, an adeno-associated viral, a retroviral, a lentiviral, baculovirus, or a herpes viral vector, using standard methods. The gene is inserted into the genome of the host cell by viral machinery where it will be expressed by the cell. The viral vector is modified so that it is replication deficient and will not produce virus, preventing viral infection in the host. The general principles for this type of therapy are known to those skilled in the art and have been reviewed in the literature (see, for example, Kohn et al., Transfusion 29:812-820, 1989; Russell, J. Gen. Virol. 81 Pt 11:2573-2604, 2000; Diaz et al., Gene Ther. 7(19): 1656-1663, 2000). Alternatively, a plasmid containing a nucleic acid encoding an antibody of interest may be administered using standard electroporation techniques (see, for example, Smith and Nordstrom, Curr. Opin. Mol. Ther. 2920:150-154, 2000).

The aforementioned genetically modified cells expressing a desired antibody or the nucleic acids encoding a desired antibody may be administered to a mammal in single or multiple doses. For any particular subject, the specific dosage regimes should be adjusted over time according to the individual need and the professional judgment of the person administering or supervising the administration of the compositions.

These methods are described further below. It is noted that these methods may be applied to any antibody-producing cell from any donor source and administered to any recipient mammal.

Production of Mouse Anti-Tetanus Antibody in Cattle

A mouse hybridoma that secretes an anti-tetanus antibody was produced using standard methods by the PEG-assisted fusion of mouse SP2/0 cells with spleen cells from a Balb/C mouse immunized with tetanus toxoid. Approximately 5×10⁸ cells of this hybridoma were injected into the dewlap, and 5×10⁸ cells were injected into the mammary region a 14 day old male calf. A blood sample taken 10 days after implantation contained mouse immunoglobulin which reacted with tetanus toxoid but did not react with BSA or a peptide derived from beta amyloid protein, based on standard ELISA analysis. Therefore, the mouse immunoglobulin generated during the growth of the xenotransplant in cattle retained its reactivity and specificity. Quantitative measurements of the level of mouse Ig in the blood of this calf using standard methods indicated the presence of approximately 300 ug of mouse antibody per liter. Assuming a total blood volume of six liters, the calf contained a total of approximately 1.8 milligrams of mouse antibody in its bloodstream.

Production of Mouse Anti-Tetanus Antibody in Fetuses

A mouse anti-tetanus antibody was also produced in the fetuses of pregnant cows. For this procedure, a fetus at day 85 of gestation and a fetus at day 90 of gestation were surgically removed from pregnant cows using standard Caesarian techniques that allowed the fetuses to be exposed so that they could be injected with hybridoma cells while they remained connected to the pregnant cows through their umbilical cords. Approximately, 3×10⁸ viable cells from the hybridoma described above that secretes an anti-tetanus antibody were injected into the peritoneal cavity of the fetus at day 85 of gestation, using ultrasound to confirm that the cells were being injected into the peritoneal cavity. For the fetus at day 90 of gestation, approximately 5×10⁸ viable cells were injected into the peritoneal cavity using standard methods as described above. The fetuses were then returned to the uterus of the pregnant cattle using standard procedures. After 13 days, the fetuses were surgically removed from the pregnant cattle and sacrificed. For each fetus, a sample of ascites fluid from the peritoneal cavity and a sample of cord blood were analyzed for the presence of mouse immunoglobulin. All of these samples contained immunoglobulin that reacted with a general sheep anti-mouse antibody, indicating the presence of mouse immunoglobulin in both the ascites fluid and blood of the fetuses. The immunoglobulin in these samples also reacted with tetanus toxoid but did not react with the negative control BSA, based on standard ELISA analysis. Therefore, the mouse immunoglobulin generated during the growth of the hybridoma cells in the fetuses retained its reactivity and specificity. Quantitative measurements of the level of mouse Ig in a cord blood sample from the fetuses indicated the presence of 50 ug antibody per mL of blood in the fetus injected with 5×10⁸ hybridoma cells and 25 ug antibody per ML of blood in the fetus injected with 3×10⁸ cells.

Inhibition of B-Cell Development in Fetuses

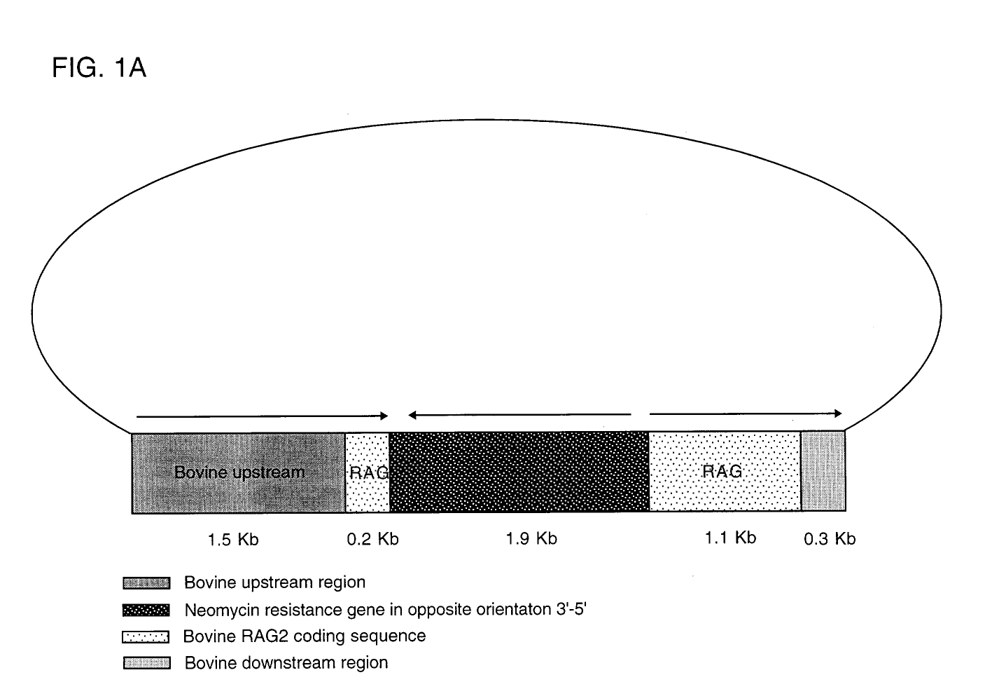

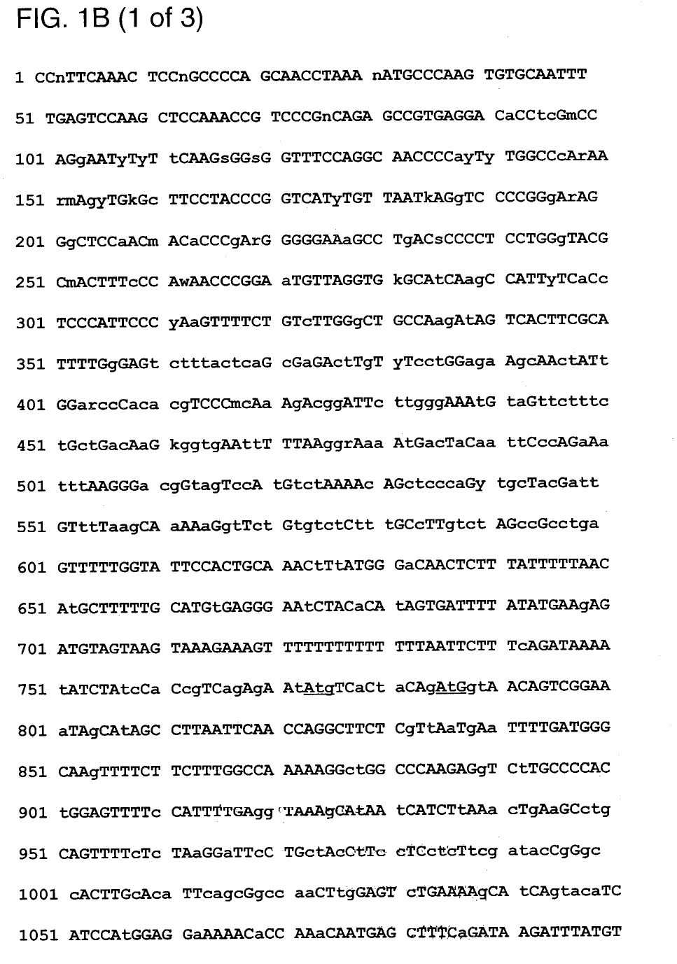

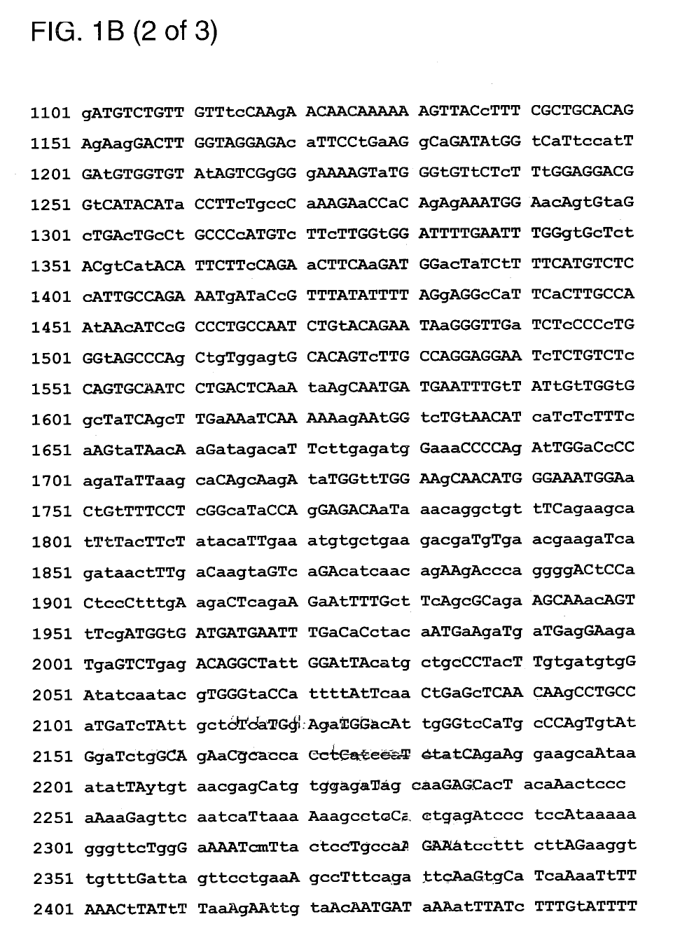

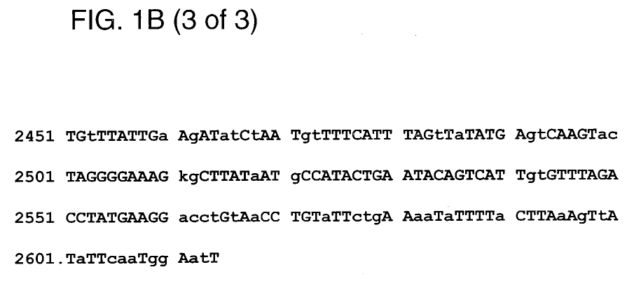

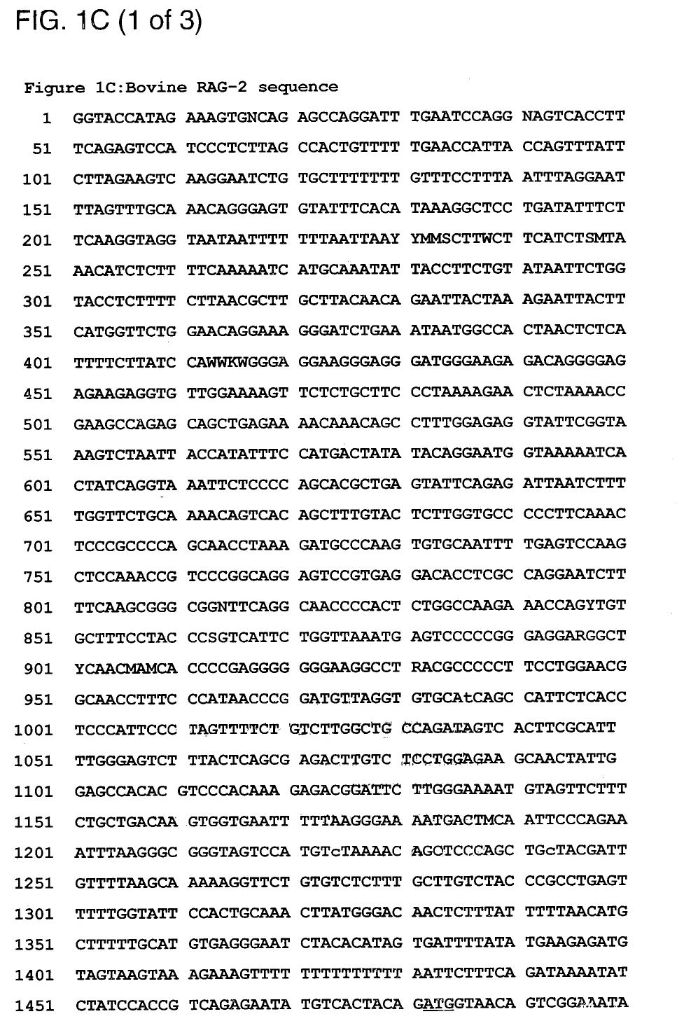

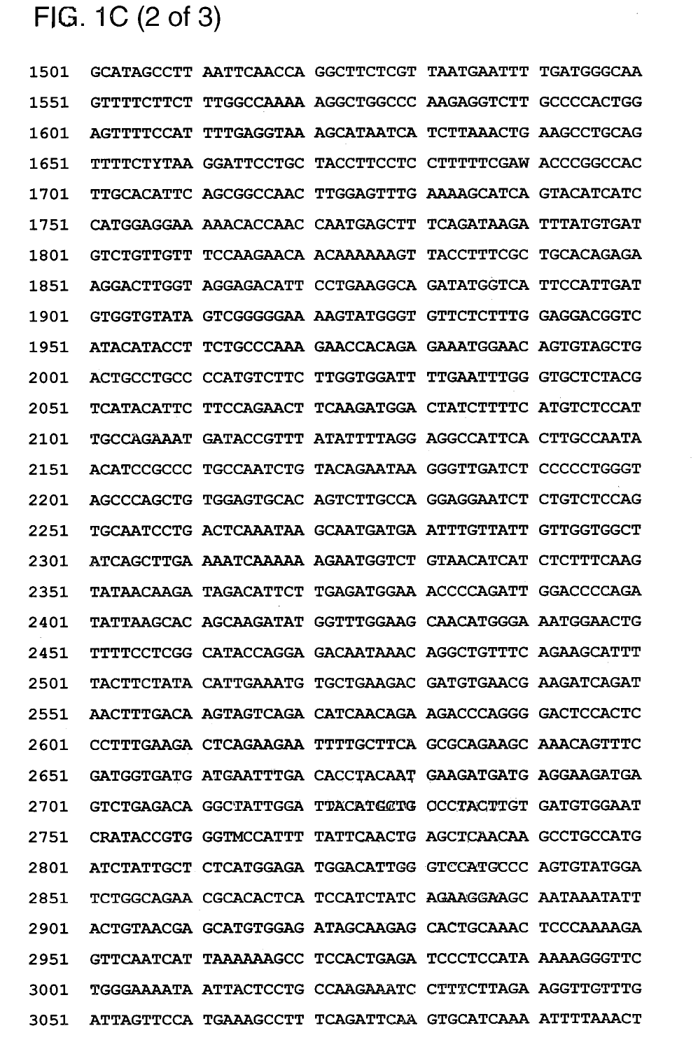

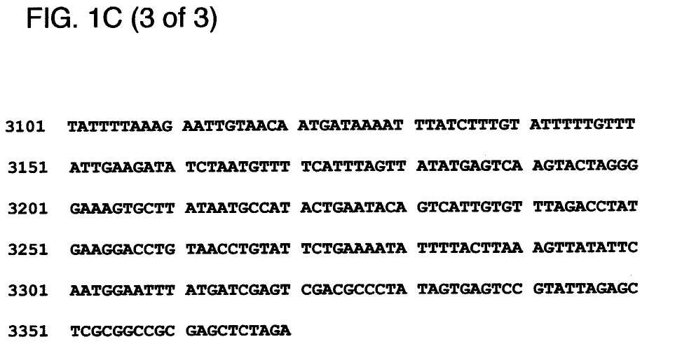

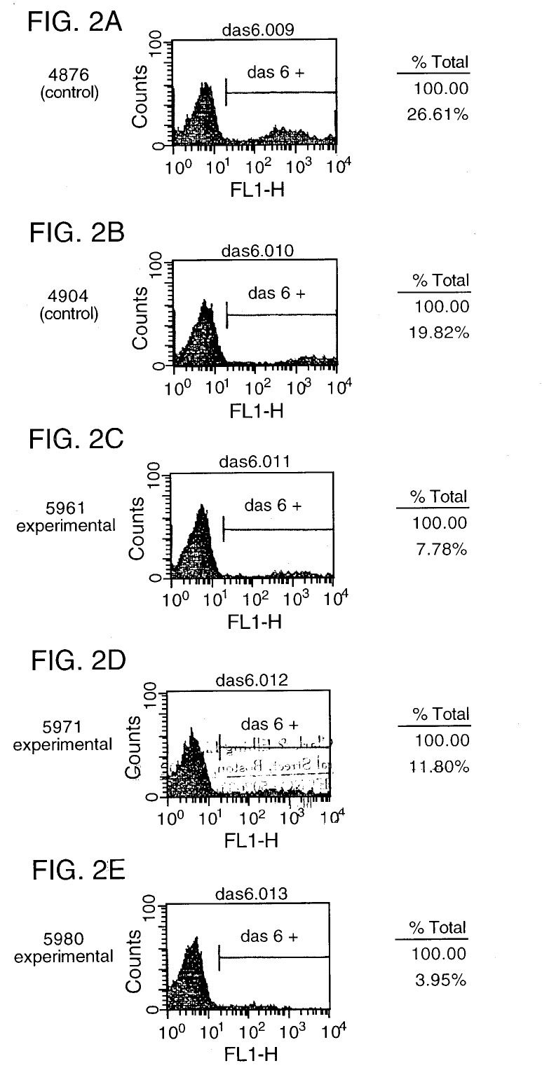

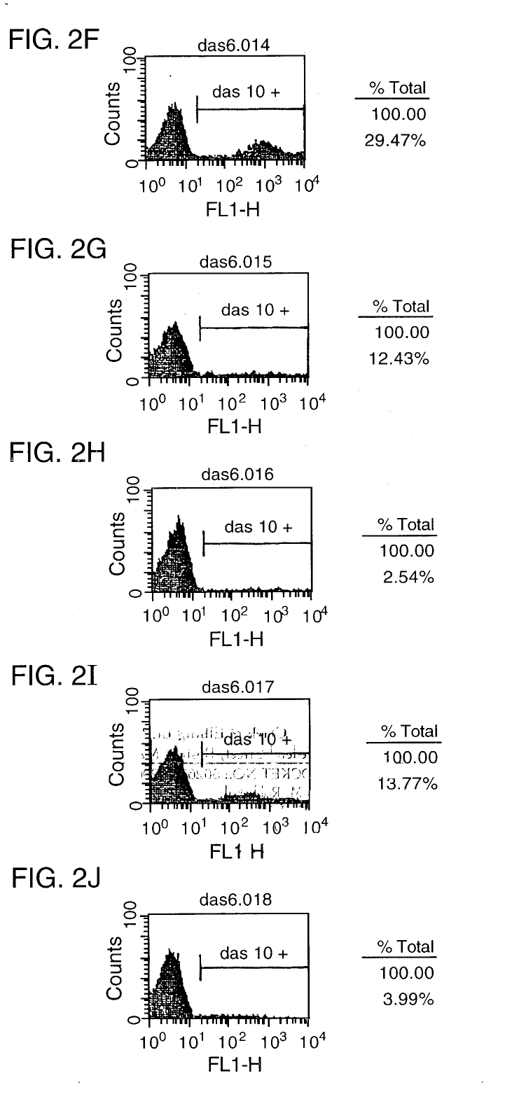

An anti-bovine IgM antibody (das6) was injected into fetuses to demonstrate the ability of this antibody to inhibit the development of B-cells in fetuses. Three fetuses at day 75 of gestation were injected with 1.5 mg of the anti-bovine IgM antibody. For this injection, the standard surgical procedure described above was used to inject the antibody into the peritoneal cavity of the fetuses. A similar injection procedure was performed on two control fetuses, which were injected with either 3×10⁷ fetal liver cells (FIGS. 2A and 2F) or 1.8×10⁷ mouse bone marrow cells (FIGS. 2B and 2G), which should not affect the number of B-cells in the fetuses. After approximately 41 days, the three experimental and two control fetuses were removed from the pregnant cows. Standard methods were used to isolate peripheral blood lymphocytes from blood samples from each of the fetuses.

To determine the percentage of peripheral blood lymphocytes that were B-cells, these lymphocytes were analyzed using standard FACS analysis for the expression of either IgM or antibody light chain molecules, which are both expressed on the surface of B-cells. As illustrated in FIG. 2A and FIG. 2B, approximately 19.82 to 26.61% of the peripheral blood lymphocytes from the control fetuses expressed IgM. In contrast, only 7.78, 11.80, or 3.95% of the peripheral blood lymphocytes from the three fetuses injected with the anti-bovine IgM antibody expressed IgM (FIGS. 2C-2E, respectively). As illustrated in FIG. 2F and FIG. 2G, approximately 12.43 to 29.47% of the peripheral blood lymphocytes from the control fetuses expressed antibody light chain molecules. For the three fetuses injected with the anti-bovine IgM antibody, 2.54, 13.77, or 3.99% of the peripheral blood lymphocytes expressed antibody light chain molecules (FIGS. 2H-2J, respectively). These results indicate that the injection of the anti-bovine IgM antibody reduced the number of B-cells in the peripheral blood of the fetuses.

Immune System Inhibition and Subsequent Transplantation of Hybridomas into Cattle for the in vivo Production of Antibodies

To inhibit an adverse immune response to a transplanted hybridoma and to the antibodies produced by the hybridoma, two to three month old calves are treated with a compound that reduces B-cell activity, a compound that reduces T-cell activity, or both compounds. This global inhibition of endogenous antibody production by B-cells may also result in fewer undesired antibodies in the serum from cattle transplanted with a hybridoma. Thus, this method may facilitate the purification of the desired antibody from blood, milk, or lymph samples.

To reduce the number and activity of circulating B-cells, a commercially available anti-bovine IgM antibody is intravenously administered to the calves. After this treatment, the number of circulating B-cells may be measured by standard fluorescence-activated cell sorting (FACS) analysis of a blood sample from the calves. In this assay, fluorescently labeled anti-bovine Ig antibodies are used to bind Ig molecules expressed on the surface of the B-cells, and the number of B-cells labeled with these antibodies is determined using FACS. The amount of antibodies secreted by B-cells is determined using a standard ELISA capture assay with an anti-bovine Ig antibody.

Preferred methods for inhibiting T-cell activity include the oral administration of cyclosporin, azathioprine, or dexamethasone to calves at a dose between approximately 5 and 15 mg/kg body weight/day. Alternatively, an antibody reactive with a T-cell surface molecule, such as commercially available anti-bovine CD3, anti-bovine CD2, or anti-bovine CD25 antibody, may be administered. For the measurement of residual T-cell activity, standard procedures may be used to isolate T-cells from a blood sample, treat them with a mitogen such as concavalin A or phytohemagglutinin, and measure cell proliferation and cytokine production. Cytokine levels may be measured using an ELISA assay with a commercially available antibody that binds the cytokine. Cell proliferation may be quantitated using FACS analysis to determine the increase in the number of T-cells in the presence of the mitogen.

Alternatively, any other immunosuppressive agent may be administered to inhibit lymphocytes. One preferred immunosuppressive agent is progesterone which may be administered in a concentration sufficient to result in approximately 100 to 500 ng or 500 to 1000 ng progesterone per mL of blood in the calf. Alternatively, an antibody that is reactive against multiple bovine lymphocytes may be generated by injecting bovine lymphocytes into rabbits or mice, using standard procedures (see, for example, Ausubel et al. Current Protocols in Molecular Biology, John Wiley & Sons, New York, 2000). Polyclonal antibodies that bind multiple bovine lymphocytes, such as B-cells, helper T-cells, and Natural Killer (NK) T-cells may be isolated from the rabbit or mice serum and administered to cattle for the inhibition of lymphocyte activity.

Blood, milk, or lymph samples may be taken at various time points, such as 1, 2, or 3 times a week, to measure the residual B-cell and T-cell activity. Preferably, the amount of antibodies secreted by B-cells, the amount of T-cell proliferation, or the amount of indicator cytokine secreted by T-cells is reduced by at least 25, 50, 75, or 90% compared to the corresponding amount in the absence of treatment to inhibit the immune system. Achieving this level of immune system inhibition may require a few days, a few weeks, or longer depending upon the daily dose of the particular compound that is administered to the calves. If necessary, larger doses or more frequent dosing schemes than those mentioned above may also be used to further reduce the level of B-cell or T-cell activity or to cause the desired reduction in activity to be achieved sooner.

When the immune system function is sufficiently inhibited, antibody-producing cells such as hybridoma cells are transplanted into the calves. For this transplantation, standard injection techniques may be used to injected calves with hybridoma cells (10⁸ cells in 4 mL of phosphate buffered saline) in one or multiple sites in the calves (e.g., a mammary region, uterus, dewlap, brisket, scrotum, testicle, or hump) to allow vascularization of the transplant and to produce minimal discomfort in the calves. If desired, the size of the resulting hybridoma in the recipient mammal or the amount of antibodies secreted by the hydribodma may be modulated by altering the number of cells that are administered. For example, to increase antibody production, more hydribodma cells may be administered. If necessary, the administration of a compound that inhibits lymphocyte activity may be maintained. Preferably, this immunosuppressive compound does not inhibit the activity of the transplanted hybridoma.

For the isolation of the antibodies secreted by the hybridoma, blood, milk, or lymph samples are taken from the calves at multiple intervals, such as every day for 1, 3, 5, 7, 14, or more days, and used in standard methods for the purification of the antibody. If desired, blood samples may also be analyzed for continued inhibition of the endogenous immune system of the calves.

Fetal Cell Transplant Procedures for Tolerization in Cattle and Subsequent Transplantation of Hybridomas into Cattle for the in vivo Production of Antibodies

In one tolerization technique, fetuses in pregnant cows are injected with a combination of mouse bone marrow cells (2 to 3 mls of 2×10⁷ cells/ml) and approximately 1-5 mg of mouse serum proteins, such as of IgM, IgD, IgG, IgE, or IgA on day 75 (2.5 months) of gestation. These cells and proteins may be obtained from commercial sources or isolated using standard cell purification techniques (such as FACS sorting) or standard protein purification techniques (see, for example, Ausubel et al., supra). The injection of mouse bone marrow cells into the fetus may be performed by exposing the gravid uterus of the pregnant cow via flank incision. This procedure is done using appropriate anesthetics and analgesics. Alternatively, the mouse cells and proteins may be administered using transvaginal ultrasound, which is minimally invasive. As these cells propagate and integrate into the fetus, tolerance to mouse cells is induced in the developing animal.

If desired, one or more fetuses may be recovered during gestation using standard Caesarian techniques to determine whether T-cells from the fetus proliferate or produce cytokines in response to mouse antigens and to determine whether B-cells secrete anti-mouse antibodies.

Alternatively, these mouse proteins or cells may be administered after birth of the calves. Preferred postnatal routes of administration include parenteral, intravenous, intraarterial, intraventricular, subcutaneous, and intramuscular administration.

The live calves may be immediately injected with a hybridoma or held until later administration, such as administration at 1, 2, 4, 6, 8, 10, 12, or 14 months of age. The calves are injected with mouse hybridoma cells (10⁸) in one or more sites, and the antibodies produced by the hybridoma cells are purified from blood, milk, or lymph samples from the calves, as described above. If desired, blood samples may also be analyzed to evaluate serum mouse Ig levels, bovine Ig levels, white blood cell levels, and other markers of animal health.

As an alternative to the above method of injecting mouse cells into a fetus to induce tolerization, mouse embryonic cells may be injected into a bovine preimplantation embryo to form a germ-line chimera (see, for example, Bradley et al., Nature 309:225-256, 1984). The preimplantation embryo may be an embryo in a pregnant cow or an embryo that is cultured in vitro and then transferred to a maternal host, as described below.

Generation of Cloned Cattle with Reduced RAG1 or RAG2 Activity and Reduced Adverse Immune Response to the Transplanted Hybridomas

Recombinase activating genes RAG1 and RAG2 are required for the recombination process which results in the generation of diverse sequences encoding antibodies and T-cell receptors. Thus, the B-cell and T-cell activities of calves may be reduced or eliminated by mutating the RAG1 or RAG2 genes. To reduce or eliminate RAG1 or RAG2 function in calves, standard molecular biology techniques are used to produce a DNA construct capable of homologously recombining with a RAG1 or RAG2 gene and inserting an antibiotic selection marker in this gene.

For the construction of a construct to “knock out” the bovine RAG2 gene, primers were designed based on the reported sequences of mouse and rabbit RAG2 and used to amplify a probe from bovine DNA. This probe was used to screen a genomic bovine DNA library for the RAG2 gene. The isolated RAG2 nucleic acid was digested with EcoRV and ligated to a commercially available plasmid pGEM-3Zf(−) which had been digested with SmaI. A portion of the sequence upstream of the RAG2 gene was removed from the ligated plasmid by digestion with HindIII to remove an approximately 5 Kb fragment and then religation to regenerate a circular plasmid. The resulting plasmid contained an insert with 1.5 Kb of the bovine sequence immediately upstream of the RAG2 coding sequence, the bovine RAG2 coding sequence, and 0.3 Kb of the bovine sequence immediately downstream of the RAG2 sequence. For the insertion of the neomycin gene to disrupt the RAG2 coding sequence, the plasmid was linearized by a single cut with BseRI and then modified so that it was blunt ended. This linearized plasmid was ligated with a blunt-ended nucleic acid fragment encoding the neomycin gene, generating a bovine RAG2 knockout construct in which the neomycin gene is inserted in the RAG2 coding sequence in the opposite orientation as the RAG2 coding sequence (FIGS. 1A and 1B). If desired, to increase the efficiency of homologous recombination between the RAG2 knockout construct and the endogenous RAG2 sequence, the RAG2 knockout construct could be modified by the insertion of additional sequence from the upstream or downstream region flanking the bovine RAG2 coding sequence to increase the length of the regions in the knockout construct that are homologous to the endogenous sequence (FIG. 1C).

For the production of a bovine RAG1 knockout construct, the above procedure may be repeated using primers designed using the reported RAG1 sequences [e.g., the Lama glama (Acession Number AF305953) or Homo sapien (Acession Number XM_{—}006283) sequence]. These standard cloning techniques may also be used to generate RAG1 and RAG2 knockout constructs for the RAG1 and RAG2 genes in any other mammal.

After generation of the RAG1 or RAG2 knockout construct, bovine fibroblast cells or any other bovine cell types are transfected with the DNA construct, and the cells with correctly targeted “knockout” inserts are selected based on their antibiotic resistance. The selected cells are then used for nuclear transfer and production of hemizygous cloned knockout fetuses. For this procedure, a selected cell or the nucleus from this cell is introduced into a recipient oocyte by any standard method, such as microinjection, electrofusion, or virus-mediated fusion (see, for example, U.S. Pat. Nos. 4,997,384 and 5,945,577). Preferably, the recipient oocyte is an enucleated metaphase II stage oocyte. At this stage, the oocyte may be activated or is already sufficiently activated to treat the introduced nucleus as it does a fertilizing sperm. For enucleation of the oocyte, part or preferably all of the DNA in the oocyte is removed or inactivated. This destruction or removal of the DNA in the recipient oocyte prevents the genetic material of the oocyte from contributing to the growth and development of the cloned mammal. One method for destroying the pronucleus of the oocyte is exposure to ultraviolet light (Gurdon, in Methods in Cell Biology, Xenopus Laevis:—Practical Uses in cell and Molecular Biology, Kay and Peng, eds., Academic Press, California, volume 36:pages 299-309, 1991). Alternatively, the oocyte pronucleus may be surgically removed by any standard technique (see, for example, McGrath and Solter, Science 220:1300-1319, 1983). In one possible method, a needle is placed into the oocyte, and the nucleus is aspirated into the inner space of the needle. The needle may then be removed from the oocyte without rupturing the plasma membrane (U.S. Pat. Nos. 4,994,384 and 5,057,420).

Either electrical or non-electrical means may be used for activating reconstituted oocytes. Electrical techniques for activating cells are well known in the art (see, for example, U.S. Pat. Nos. 4,994,384 and 5,057,420). Non-electrical means for activating cells may include any method known in the art that increases the probability of cell division. Examples of non-electrical means for activating an oocyte include incubating the oocyte in the presence of ethanol; inositol trisphosphate; Ca⁺⁺ ionophore and a protein kinase inhibitors; a protein synthesis inhibitor; phorbol esters; thapsigargin, or any component of sperm. Other non-electrical methods for activation include subjecting the oocyte to cold shock or mechanical stress. Alternatively, one to three hours after nuclear transfer, oocytes may be incubated for approximately six hours in medium containing Sr²⁺ to activate them. If the donor cell or donor nucleus has a DNA content of 2n (where “n” is the number of chromosomes found in the normal haploid chromosome set of a mammal of a particular genus or species), the reconstituted oocyte may also be activated in the presence of cytochalasin B, or cytochalasin B may be added immediately after activation to prevent polar body extrusion and chromosome loss (Wakayama et al., PNAS 96:14984-14989, 1999; Wakayama et al., Nature Genetics 24:108-109, 2000). For cows, the oocyte activation period generally ranges from about 16-52 hours or preferably about 28-42 hours. For other mammals, the preferred length of activation may vary.

After activation, the oocyte is placed in culture medium for an appropriate amount of time to allow development of the resulting embryo. At the two cell stage or a later stage, the embryo is transferred into a foster recipient female. For bovine species, the embryos are typically cultured to the blastocyst stage (e.g., for approximately 6-8 days) before being transferred to maternal hosts. For other cloned animals, an appropriate length for in vitro culturing is known by one skilled in the art or may be determined by routine experimentation.

Methods for implanting embryos into the uterus of a mammal are also well known in the art. Preferably, the developmental stage of the embryo is correlated with the estrus cycle of the host mammal. Embryos from one species may be placed into the uterine environment of an animal from another species. For example, bovine embryos can develop in the oviducts of sheep (Stice and Keefer, Biology of Reproduction 48: 715-719, 1993). Any cross-species relationship between embryo and uterus may be used in the methods of the invention.

A fetus is recovered from the host using standard Caesarian procedures, and a cell line is derived from the fetus. The fetal cell line is then transfected with the DNA construct described above to knockout the second allele of the RAG1 or RAG2 gene to produce a homozygous cell line. This cell line is used for another round of nuclear transfer to produce a fetus, as described above. A homozygous fetal cell line is then derived and used to produce cloned knockout calves.

These calves are then used as recipient mammals for the transplantation of hybridoma cells that produce an antibody of interest. For example, calves may be injected with hybridoma cells, as described previously, at three months of age or at any other age. Blood, milk, or lymph samples containing this desired antibody are removed from the calves for isolation of the antibody. If desired, blood samples may also be analyzed to evaluate serum mouse Ig levels, bovine Ig levels, white blood cell levels, and other markers of animal health.

Production of Antibodies in Goats

Other preferred recipient mammals for producing antibodies of interest include goats, such as pigmy goats. Because of their large size and long life-span, goats may be used to produce vast quantities of antibodies. For the production of antibodies in goats, the same procedures as described above for cattle may be used. For example, goats may be injected with any of the antibody-producing cells of the present invention in one or multiple sites (e.g., a mammary region, uterus, scrotum, or testicle). The antibodies produced by the transplanted cells may be isolated from blood, milk, or lymph samples using standard procedures.

To reduce any adverse immune response to the transplanted cells or to the resulting antibodies, the goats may be optionally treated with a compound that reduces B-cell activity (e.g., an anti-goat IgM antibody), a compound that reduces T-cell activity (e.g., cyclosporin, azathioprine, dexamethasone, an anti-goat CD3 antibody, an anti-goat CD2 antibody, or an anti-goat CD25 antibody), or both classes of compounds. These anti-goat antibodies may be obtained from commercial sources or generated using standard methods by injecting the corresponding goat antigens into mice or rabbits (see, for example, Ausubel et al., supra). These compounds may be administered before, during, or after the administration of the antibody-producing cells. These compounds may also be administered during the normal developmental period of the goat immune system to inhibit the development of B-cells and T-cells in the goats.

Additionally, any adverse immune response to the foreign antibody-producing cells or foreign antibodies may be reduced by exposing the goats to proteins or cells from the same genus or species as the subsequently transplanted antibody-producing cells. In particular, these proteins or cells may be administered during the developmental stage of the goats' immune system to induce tolerization to similar proteins or cells. If the cells are administered during the embryonic stage of a goat, a germ-line chimeric mammal may be produced that contains cells from a goat and cells from another genus or species.

Moreover, goats containing a mutation that reduces or eliminates RAG1 or RAG2 activity may be generated as described above for the generation of cloned cattle with reduced RAG1 or RAG1 activity. These goats may have reduced B-cell and T-cell activity and thus may have a decreased immune response against the foreign antibody-producing cells and the antibodies produced by these cells.

Standard cloning methods may also be used to produce multiple cloned goats that each have an identical or a substantially identical genome as that of the donor source from which the antibody-producing cells are derived (see, for example, U.S. Pat. Nos. 5,945,577 and 6,011,197). These cloned goats may also have increased tolerance for the antibody-producing cells and the resulting antibodies.

Production of Antibodies in Pigs

As described in the examples above for cattle and goats, standard methods may also be used to produce antibodies of interest in pigs, which are also desirable recipient mammals because of their large size and long life-span. Pigs may be injected with any of the antibody-producing cells of the present invention in one or multiple sites (e.g., a mammary region, uterus, scrotum, or testicle), and the resulting antibodies may be isolated from blood, milk, or lymph samples. Before, during, or after the administration of the antibody-producing cells, the pigs may be optionally treated with a compound that reduces B-cell activity (e.g., an anti-pig IgM antibody), a compound that reduces T-cell activity (e.g., cyclosporin, azathioprine, dexamethasone, an anti-pig CD3 antibody, an anti-pig CD2 antibody, or an anti-pig CD25 antibody), or both classes of compounds. These anti-pig antibodies may be obtained from commercial sources or generated using standard methods by injecting the corresponding pig antigens into mice or rabbits (see, for example, Ausubel et al., supra). These compounds may also be administered during the normal developmental period of the pig immune system to inhibit the development of B-cells and T-cells in the pigs.

Any adverse immune response to the foreign antibody-producing cells or foreign antibodies may also be reduced by administering proteins or cells from the same genus or species as the subsequently transplanted antibody-producing cells. Preferably, these proteins or cells are administered during the developmental stage of the pigs' immune system. Additionally, pigs containing a mutation that reduces or eliminates RAG1 or RAG2 activity and thus have a decreased immune response against the foreign cells and proteins may be generated as described above for the generation of cloned cattle or goats with reduced RAG1 or RAG1 activity. Standard cloning methods may also be used to produce multiple cloned pigs that each have an identical or a substantially identical genome as that of the donor source from which the antibody-producing cells are derived and thus have increased tolerance for the antibody-producing cells and the resulting antibodies (see, for example, U.S. Pat. Nos. 5,945,577 and 6,011,197).

Production of Antibodies in Horses

In addition to their large size and long life-span, horses are preferred recipient mammals for use in the methods of the present invention because of the relative ease by which blood samples may be obtained from horses. As described in the previous examples, horses may be injected with any of the antibody-producing cells of the present invention in one or multiple sites (e.g., a mammary region, uterus, scrotum, or testicle), and the resulting antibodies may be isolated from blood, milk, or lymph samples. Before, during, or after the administration of the antibody-producing cells, the horses may be optionally treated with a compound that reduces B-cell activity (e.g., an anti-horse IgM antibody), a compound that reduces T-cell activity (e.g., cyclosporin, azathioprine, dexamethasone, an anti-horse CD3 antibody, an anti-horse CD2 antibody, or an anti-horse CD25 antibody), proteins or cells from the same genus or species as the subsequently transplanted antibody-producing cells, or a combination thereof. Additionally, horses that contain a mutation that reduces or eliminates RAG1 or RAG2 activity or that have an identical or a substantially identical genome as that of the donor source from which the antibody-producing cells are derived may be generated as described above for the generation of other cloned mammal (see, for example, U.S. Pat. Nos. 5,945,577 and 6,011,197).

Production of Antibodies in Other Mammals

Any of the above methods for the production of antibodies of interest may be performed using any other mammal. Because standard injection techniques are used to administer the antibody-producing cells of the present invention to a mammal, these techniques may be generally applied to any mammal. Similarly, the immunosuppression methods of administering a compound that inhibits B-cell activity, T-cell activity, or any other innnate or adaptive immune system activity may be used for any recipient mammal. Proteins or cells from the same genus or species as the subsequently transplanted antibody-producing cells may also be administered to any mammal. Additionally, general methods have been described for the cloning of any non-human mammal (see, for example, U.S. Pat. Nos. 5,945,577 and 6,011,197). Thus, these methods may be used to generate a variety of cloned mammals with reduced expression or activity of IgM, IgD, IgG, IgE, IgA, RAG1, or RAG2 or with an identical or a substantially identical genome as that of the donor source from which the antibody-producing cells are derived.

Optional Evaluation of Pain, Discomfort, and Overall Health of Mammals with Transplanted Antibody-Producing Cells

If desired, the recipient mammals used in the methods of the invention may be evaluated for signs of pain or discomfort from the transplanted antibody-producing cells. Standard clinical chemistry analyses may be performed on blood samples from the mammals. White blood cell counts and red blood cell counts may also be determined and compared to clinical norms. Respiration, heart rate, and temperature are determined on a weekly basis. Food and water intake is measured. In addition, daily behavioral observations are made and recorded on a score chart. The score chart includes observations of activity, watery or dry eyes and nose, and signs of diarrhea. Weekly estimates of transplant size are made, signs of tenderness in response to palpation are recorded, and notes are made about transplant induced restrictions or alterations to movement by the mammals. The results may be graphed to evaluate trends and to indicate need for veterinary intervention or euthanasia. If recipient mammals show signs of pain or discomfort from the transplant, they may be euthanized. The transplant and the scarified animal may be evaluated to determine the cause of pain or discomfort.

Other Embodiments

From the foregoing description, it will be apparent that variations and modifications may be made to the invention described herein to adopt it to various usages and conditions. Such embodiments are also within the scope of the following claims.

All publications mentioned in this specification are herein incorporated by reference to the same extent as if each independent publication or patent application was specifically and individually indicated to be incorporated by reference.

What is claimed is:

1. A method of producing antibodies, said method comprising the steps of:
(a) administering an antibody-producing cell from a donor source to a non-rodent, non-human recipient mammal in a site other than the peritoneal cavity; and
(b) isolating the antibodies produced by said antibody-producing cell from said recipient mammal.

2. A method of producing antibodies, said method comprising the steps of:
(a) administering an antibody-producing cell from a donor source to a non-rodent, non-human recipient mammal during the embryonic or fetal stage of said recipient mammal; and
(b) isolating the antibodies produced by said antibody-producing cell from said recipient mammal during the embryonic, fetal, or postnatal stage of said recipient mammal.

3. The method of claim 2, wherein said antibody-producing cell is administered to a mammary gland, uterus, dewlap, brisket, scrotum, testicle, or hump of said recipient mammal.

4. The method of claim 2, further comprising administering a compound that inhibits B-cell activity to said recipient mammal in an amount sufficient to reduce said B-cell activity in said recipient mammal.

5. The method of claim 2, further comprising administering a compound that inhibits T-cell activity to said recipient mammal in an amount sufficient to reduce said T-cell activity in said mammal.

6. The method of claim 2, further comprising administering a cell of the same genus or species as said donor source to said recipient mammal during the normal period of immune system development of said recipient mammal.

7. The method of claim 2, further comprising administering a protein from a cell, embryo, fetus, or mammal of the same genus or species as said donor source to said recipient mammal during the normal period of immune system development of said recipient mammal.

8. The method of claim 2, wherein said recipient mammal is a chimeric mammal that comprises both cells of the same genus or species as said donor source and cells of a different genus or species.

9. The method of claim 2, wherein said recipient mammal comprises a mutation that reduces or eliminates the expression or activity of IgM, IgD, IgG, IgE, IgA, RAG1, or RAG2.

10. The method of claim 2, wherein said recipient mammal is a cow, sheep, goat, buffalo, rabbit, or pig.

11. A method of transplanting an antibody-producing cell into a recipient mammal, said method comprising the steps of:
(a) tolerizing said recipient mammal to said antibody-producing cell or to the antibodies produced by said antibody-producing cell; and
(b) administering said antibody-producing cell to said recipient mammal.

12. The method of claim 11, wherein said antibody-producing cell is administered to a mammary gland, uterus, dewlap, brisket, scrotum, testicle or hump of said recipient mammal.

13. A method of transplanting an antibody-producing cell into a recipient mammal, said method comprising the steps of:
(a) suppressing the immune system of said recipient mammal; and
(b) administering an antibody-producing cell to said recipient mammal.

14. The method of claim 13, wherein said immunosuppression step comprises administering a compound that inhibits B-cell activity to said recipient mammal in an amount sufficient to reduce said B-cell activity in said recipient mammal.

15. The method of claim 13, wherein said immunosuppression step comprises administering a compound that inhibits T-cell activity to said recipient mammal in an amount sufficient to reduce said T-cell activity in said recipient mammal.

16. A method of transplanting an antibody-producing cell into a non-human recipient mammal, said method comprising administering an antibody-producing cell to said recipient mammal, wherein said recipient mammal is a chimeric mammal that comprises both cells of the same genus or species as said antibody-producing cell and cells of a different genus or species as said antibody-producing cell.

17. A method of transplanting an antibody-producing cell into a non-human recipient mammal, said method comprising administering an antibody-producing cell to said recipient mammal, wherein said recipient mammal comprises a mutation that reduces or eliminates the expression or activity of IgM, IgD, IgG, IgE, IgA, RAG1, or RAG2.

18. A method of treating or preventing a disease, disorder, or infection in a mammal, said method comprising the steps of:
(a) inserting a nucleic acid encoding a desired antibody into a cell obtained from said mammal, thereby forming an antibody-producing cell; and
(b) administering said antibody-producing cell to said mammal.

19. The method of claim 18, further comprising inserting a nucleic acid encoding an oncogene prior to step (a) and removing said nucleic acid encoding an oncogene prior to step (b).

20. The method of claim 18, wherein said mammal is a human.
